(19) United States
(12) Patent Application Publication
(10) Pub. No.: US 2022/0175701 A1
(43) Pub. Date: Jun. 9, 2022

(54) TREATMENT OF MAJOR DEPRESSIVE DISORDER AND SUICIDAL IDEATIONS THROUGH STIMULATION OF HIPPOCAMPAL NEUROGENESIS UTILIZING PLANT-BASED APPROACHES

(71) Applicant: THERAPEUTIC SOLUTIONS INTERNATIONAL, INC., OCEANSIDE, CA (US)

(72) Inventors: Thomas Ichim, Oceanside, CA (US); Timothy G. Dixon, Oceanside, CA (US); James Veltmeyer, Oceanside, CA (US); Kalina O'Connor, Oceanside, CA (US)

(73) Assignee: THERAPEUTIC SOLUTIONS INTERNATIONAL, INC., OCEANSIDE, CA (US)

(21) Appl. No.: 17/545,301

(22) Filed: Dec. 8, 2021

Related U.S. Application Data
(60) Provisional application No. 63/122,862, filed on Dec. 8, 2020.

(51) Int. Cl. A61K 31/138; A61K 36/235; A61K 36/82; A61K 31/09; A61K 31/095; A61K 45/06

(52) U.S. Cl. CPC A61K 31/138; A61K 36/235; A61K 45/06; A61K 31/09; A61K 31/095; A61K 36/82

(57) ABSTRACT

Disclosed are means and methods of treating major depressive disorder and/or other disorders that predispose to suicide by administration of nutraceutical means, wherein said nutraceuticals are administered at a frequency and/or concentration sufficient to induce proliferation of endogenous neural progenitor cells. In one embodiment said nutraceuticals are comprised of green tea extract, and/or Nigella sativa, and/or pterostilbene, and/or sulforaphane. In some embodiment's nutraceutical compositions are utilized to overcome treatment resistant of currently used antidepressants.

### CROSS-REFERENCE TO RELATED APPLICATIONS

This application claims the benefit of priority to U.S. Provisional Application Ser. No. 63/122,862, filed Dec. 8, 2020, the contents of which are incorporated herein by reference.

### FIELD OF THE INVENTION

The invention pertains to the area of psychiatry, more specifically, the invention relates to the utilization of chemicals useful for stimulation of neurogenesis. More specifically the invention relates to the use of neurogenesis for treatment of major depressive disorder.

### BACKGROUND OF THE INVENTION

MDD is a condition associated with depression, lack of interest, anhedonia, fear, feelings of worthlessness, weight loss, insomnia, and inability to maintain concentration. It is believed that MDD has 12-month and lifetime prevalence of 10.4% and 20.6%, respectively [1]. This disease is also often a life-threatening illness with suicide as cause of death for an estimated 10% of patients with severe MDD. Current treatments of MDD include the use of antidepressants, sleep deprivation, electroconvulsive therapy and ketamine.

An inflammatory basis for MDD has been proposed by several investigators. Initial findings included elevated inflammatory cytokines in the blood of patients. One of the first studies examined data collected from 3024 well-functioning older persons, 70-79 years of age. Depressed mood was defined as a Center for Epidemiologic Studies Depression scale score of 16 or higher. Plasma concentrations of interleukin (IL)-6, tumor necrosis factor (TNF)-alpha, and C-reactive protein (CRP) were measured. Compared with the 2879 nondepressed subjects, the 145 persons with depressed mood had higher median plasma levels of IL-6, TNF-alpha, and CRP. After adjustment for health and demographic variables, depressed mood was especially prevalent among persons who had a high (above median) plasma level for at least two of the inflammatory markers [2]. Numerous other studies have demonstrated upregulation of plasma IL-6 in patients with MDD [3-32].

Interestingly, pointing to a pathological role of IL-6 are studies in which elevations of this acute phase protein are associated with resistance to psychiatric therapy of MDD. In one study, plasma concentrations of IL-6 was assessed in unmedicated, medically stable patients with MDD (n=98) and varying numbers of adequate antidepressant treatment trials in the current depressive episode as measured by the Massachusetts General Hospital Antidepressant Treatment Response Questionnaire. Covariates including age, sex, race, education, body mass index (BMI) and severity of depression were included in statistical models where indicated. The investigators found a significant relationship between number of failed treatment trials and inflammatory markers including IL-6 [33]. In another study demonstrating a role of IL-6 in treatment resistance, twenty-nine patients who suffered from a current major depressive episode diagnosed using DSM-IV-TR criteria and were scheduled to undergo ECT at an academic referral center had levels inflammatory cytokines tested including IL-6 and severity of depressive symptoms (Montgomery-Asberg Depression Rating Scale [MADRS]) were prospectively evaluated before ECT treatment, after the second ECT session, and again at the completion of the index treatment series. The investigators reported that in multivariate analyses, higher levels of IL-6 at baseline, but not other inflammatory markers or clinical variables, were associated with lower end-of-treatment MADRS score [34].

### SUMMARY

Preferred methods include treating major depressive disorder comprising stimulation of adult neurogenesis in a mammal.

Preferred method include embodiments wherein said adult neurogenesis is stimulated by administration of human chorionic gonadotropin.

Preferred method include embodiments wherein said adult neurogenesis is stimulated by administration of a nutraceutical composition.

Preferred method include embodiments wherein said nutraceutical composition contains a mixture containing one or more of: a) pterostilbene; b) sulforaphane; c) green tea extract and; d) nigella sativa.

Preferred method include embodiments wherein said nutraceutical composition is QuadraMune™.

v wherein said nutraceutical is administered together with an anti-inflammatory agent.

Preferred method include embodiments wherein said anti-inflammatory agent is minocycline.

Preferred method include embodiments wherein the drug chlorpromazine is added said nutraceutical.

Preferred method include embodiments wherein the drug haloperidol is added said nutraceutical.

Preferred method include embodiments wherein the drug perphenazine is added said nutraceutical.

Preferred method include embodiments wherein the drug perphenazine is added said nutraceutical.

Preferred method include embodiments wherein the drug fluphenazine is added said nutraceutical.

Preferred method include embodiments wherein the drug clozapine is added said nutraceutical.

Preferred method include embodiments wherein the drug risperidone is added said nutraceutical.

Preferred method include embodiments wherein the drug olanzapine is added said nutraceutical.

Preferred method include embodiments wherein the drug quetiapine is added said nutraceutical.

Preferred method include embodiments wherein the drug ziprasidone is added said nutraceutical.

Preferred method include embodiments wherein the drug aripiprazole is added said nutraceutical.

Preferred method include embodiments wherein the drug paliperidone is added said nutraceutical.

Preferred method include embodiments wherein said nutraceutical is administered together with an inhibitor of NF-kappa B.

Preferred method include embodiments wherein said NF-kappa B inhibitor is selected from a group comprising of: NF-kappa B activity is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

### BRIEF DESCRIPTION OF THE DRAWINGS

FIG. 1 is a bar graph showing the results of stressed mice that were treated with Fluoxetine or QuadraMune™ daily. Assessment of endogenous neurogenesis was performed by administering BRDU and assessment of incorporation by histology.

### DETAILED DESCRIPTION OF THE INVENTION

The invention provides means of stimulating endogenous neurogenesis by administration of nutraceutical compounds alone or in combination with anti-inflammatories and/or other therapeutic agents.

In one embodiment the invention teaches administration of QuadraMune™ as a means of treating major depressive disorder and/or overcoming resistance to therapeutic effects of antidepressants in treatment of major depressive disorder. In some embodiments probiotics are administered to augment therapeutic efficacy.

QuadraMune™ or ingredients thereof, alone, or in combination, are disclosed by the current invention for treatment of schizophrenia and/or suicidal ideations. QuadraMune™ is comprised of Nigella sativa, Sulforaphane, Pterostilbene, and EGCG.

Pterostilbene (trans-3,5-dimethoxy-4-hydroxystilbene) is a natural polyphenolic compound, primarily found in fruits, such as blueberries, grapes, and tree wood. It has been demonstrated to possess potent antioxidant and anti-inflammatory properties. It is a dimethylated analog of resveratrol which is found in blueberries [35], and is believed to be one of the active ingredients in ancient Indian Medicine [36]. The pterostilbene molecule is structurally similar to resveratrol, the antioxidant found in red wine that has comparable anti-inflammatory, and anticarcinogenic properties; however, pterostilbene exhibits increased bioavailability due to the presence of two methoxy groups which cause it to exhibit increased lipophilic and oral absorption [37-41]. In animal studies, pterostilbene was shown to have 80% bioavailability compared to 20% for resveratrol making it potentially advantageous as a therapeutic agent [37].

We have demonstrated the pterostilbene administered in the form of nanostilbene in cancer patients results in increased NK cell activity, as well as interferon gamma production. Additionally, pterostilbene has shown to inhibit inflammatory cytokines associated with ARDS. For example, studies have demonstrated inhibition of interleukin-1 [42], interleukin-6 [43, 44], interleukin-8 [45], and TNF-alpha [46], by pterostilbene.

It is interesting to note that numerous studies have demonstrated endothelial protective effects of pterostilbene. For example, Zhang et al. investigated the anti-apoptotic effects of pterostilbene in vitro and in vivo in mice. Exposure of human umbilical vein VECs (HUVECs) to oxLDL (200 μg/ml) induced cell shrinkage, chromatin condensation, nuclear fragmentation, and cell apoptosis, but pterostilbene protected against such injuries. In addition, PT injection strongly decreased the number of TUNEL-positive cells in the endothelium of atherosclerotic plaque from apoE(−/−) mice. OxLDL increased reactive oxygen species (ROS) levels, NF-κB activation, p53 accumulation, apoptotic protein levels and caspases-9 and -3 activities and decreased mitochondrial membrane potential (MMP) and cytochrome c release in HUVECs. These alterations were attenuated by pretreatment. Pterostilbene inhibited the expression of lectin-like oxLDL receptor-1 (LOX-1) expression in vitro and in vivo. Cotreatment with PT and siRNA of LOX-1 synergistically reduced oxLDL-induced apoptosis in HUVECs. Overexpression of LOX-1 attenuated the protection by pterostilbene and suppressed the effects of pterostilbene on oxLDL-induced oxidative stress. Pterostilbene may protect HUVECs against oxLDL-induced apoptosis by downregulating LOX-1-mediated activation through a pathway involving oxidative stress, p53, mitochondria, cytochrome c and caspase protease [47]. Endothelial protection by pterostilbene [48, 49], and its analogue resveratrol are well known [50, 51].

The seeds of Kalonji (Nigella sativa Linneaus) are used by the Egyptian public as carminative and flavoring agents in bread and across the Middle East for a variety of food purposes [52]. This black cumin herb goes by many different names. For example, in old Latin it is called as ‘Panacea’ meaning ‘cure all’ while in Arabic it is termed as ‘Habbah Sawda’ or ‘Habbat el Baraka’ translated as ‘Seeds of blessing’. In India it is called as Kalonji while in China it is referred as Hak Jung Chou. The plant belongs to the Ranunculaceae family of flowering plants and genus of about 14 species including Nigella arvensis, Nigella ciliaris, Nigella damascene, Nigella hispanica, Nigella integrifolia, Nigella nigellastrum, Nigella orientalis and Nigella sativa, respectively. Among these, Nigella sativa is the species most exhaustively investigated for therapeutic purposes although other species have also been implicated for therapeutic uses [53]. Generally therapeutic properties of Nigella sativa have including antimicrobial [54-60], antiviral [61-64], antifungal [65, 66], anti-asthmatic/antiairway inflammation [67-81], anti-oxidant [82-86], anti-diabetic [87-97], anti-cancerous [98-113], hepatoprotective [114-127], cardioprotective [128-142], neuroprotective [143-180], renoprotective [181-194], anti-coagulant [195, 196], protects from sepsis [197-199], protects the endothelium [200-204], anti-inflammatory [205-217], and immune stimulatory [197, 218-228].

First. Taking Kalonji increases the potency of the immune system [229, 230]. Specifically, it has been shown that kalonji activates the natural killer cells of the immune system. Natural killer cells, also called NK cells are the body's first line of protection against viruses. It is well known that patients who have low levels of NK cells are very susceptible to viral infections. Kalonji has been demonstrated to increase NK cell activity. In a study published by Dr. Majdalawieh from the American University of Sharjah, Sharjah, United Arab Emirates [222], it was shown that the aqueous extract of Nigella sativa significantly enhances NK cytotoxic activity. According to the authors, this supports the idea that NK cell activation by Kalonji can protect not only against viruses, but may also explain why some people report this herb has activity against cancer. It is known that NK cells kill virus infected cells but also kill cancer cells. There are several publications that show that Kalonji has effects against cancer [98, 100, 109, 231-242].

Second. Kalonji suppresses viruses from multiplying. If the virus manages to sneak past the immune system and enters the body, studies have shown that Kalonji, and its active ingredients such as thymoquinone, are able to directly stop viruses, such as coronaviruses and others from multiplying. For example, a study published from University of Gaziantep, in Turkey demonstrated that administration of Kalonji extract to cells infected with coronavirus resulted in suppression of coronavirus multiplication and reduction of pathological protein production [243]. Antiviral activity of Kalonji was demonstrated in other studies, for example, for example, viral hepatitis, and others [244].

Third. Kalonji protects the lungs from pathology. Kalonji was also reported by scholars to possess potent anti-inflammatory effects where its active ingredient thymoquinone suppressed effectively the lipopolysaccharide-induced inflammatory reactions and reduced significantly the concentration of nitric oxide, a marker of inflammation [245]. Moreover, Kalonji has been proven to suppress the pathological processes through blocking the activities of IL-1, IL-6, nuclear factor-κB [246], IL-1β, cyclooxygenase-1, prostaglandin-E2, prostaglandin-D2 [247], cyclocoxygenase-2, and TNF-α [248] that act as potent inflammatory mediators and were reported to play a major role in the pathogenesis of Coronavirus infection.

Fourth. Kalonji protects against sepsis/too much inflammation. In peer reviewed study from King Saud University, Riyadh, Saudi Arabia, scientists examined two sets of mice (n=12 per group), with parallel control groups, were acutely treated with thymoquinone (ingredient from Kalonji) intraperitoneal injections of 1.0 and 2.0 mg/kg body weight, and were subsequently challenged with endotoxin Gram-negative bacteria (LPS 0111:B4). In another set of experiments, thymoquinone was administered at doses of 0.75 and 1.0 mg/kg/day for three consecutive days prior to sepsis induction with live Escherichia coli. Survival of various groups was computed, and renal, hepatic and sepsis markers were quantified. Thymoquinone reduced mortality by 80-90% and improved both renal and hepatic biomarker profiles. The concentrations of IL-la with 0.75 mg/kg thymoquinone dose was 310.8±70.93 and 428.3±71.32 pg/ml in the 1 mg/kg group as opposed to controls (1187.0±278.64 pg/ml; P<0.05). Likewise, IL-10 levels decreased significantly with 0.75 mg/kg thymoquinone treatment compared to controls (2885.0±553.98 vs. 5505.2±333.96 pg/ml; P<0.01). Mice treated with thymoquinone also exhibited relatively lower levels of TNF-α and IL-2 (P values=0.1817 and 0.0851, respectively). This study gives strength to the potential clinical relevance of thymoquinone in sepsis-related morbidity and mortality reduction and suggests that human studies should be performed [249].

Sulforaphane [1-isothiocyanato-4-(methylsulfinyl)-butane], an isothiocyanate, is a chemopreventive photochemical which is a potent inducer of phase II enzyme involved in the detoxification of xenobiotics [250]. Sulforaphane is produced from the hydrolysis of glucoraphanin, the most abundant glucosinolate found in broccoli, and also present in other Brassicaceae [251]. Numerous studies have reported prevention of cancer [252-256], as well as cancer inhibitory properties of sulforaphane [257-262]. Importantly, this led to studies which demonstrated anti-inflammatory effects of this compound.

One of the fundamental features of inflammation is production of TNF-alpha from monocytic lineage cells. Numerous studies have shown that sulforaphane is capable of suppressing this fundamental initiator of inflammation, in part through blocking NF-kappa B translocation. For example, Lin et al. compared the anti-inflammatory effect of sulforaphane on LPS-stimulated inflammation in primary peritoneal macrophages derived from Nrf2 (+/+) and Nrf2 (−/−) mice. Pretreatment with sulforaphane in Nrf2 (+/+) primary peritoneal macrophages potently inhibited LPS-stimulated mRNA expression, protein expression and production of TNF-alpha, IL-1beta, COX-2 and iNOS. HO-1 expression was significantly augmented in LPS-stimulated Nrf2 (+/+) primary peritoneal macrophages by sulforaphane. Interestingly, the anti-inflammatory effect was attenuated in Nrf2 (−/−) primary peritoneal macrophages. We concluded that SFN exerts its anti-inflammatory activity mainly via activation of Nrf2 in mouse peritoneal macrophages [263]. In a similar study, LPS-challenged macrophages were observed for cytokine production with or without sulforaphane pretreatment. Macrophages were pre-incubated for 6 h with a wide range of concentrations of SFN (0 to 50 μM), and then treated with LPS for 24 h. Nitric oxide (NO) concentration and gene expression of different inflammatory mediators, i.e., interleukin (IL)-6, tumor necrosis factor (TNF)-α, and IL-1β, were measured. sulforaphane neither directly reacted with cytokines, nor with NO. To understand the mechanisms, the authors performed analyses of the expression of regulatory enzyme inducible nitic oxide synthase (iNOS), the transcription factor NF-E2-related factor 2 (Nrf2), and its enzyme heme-oxygenase (HO)-1. The results revealed that LPS increased significantly the expression of inflammatory cytokines and concentration of NO in non-treated cells. sulforaphane was able to prevent the expression of NO and cytokines through regulating inflammatory enzyme iNOS and activation of Nrf2/HO-1 signal transduction pathway [264]. These data are significant because studies have shown both TNF-alpha but also interleukin-6 are involved in pathology of COVID-19 [265-275]. The utilization of sulforaphane as a substitute for anti-IL-6 antibodies would be more economical and potentially without associated toxicity. Other studies have also demonstrated ability of sulforaphane to suppress IL-6 [276-278]. Interestingly, a clinical study was performed in 40 healthy overweight subjects (ClinicalTrials.gov ID NCT 03390855). Treatment phase consisted on the consumption of broccoli sprouts (30 g/day) during 10 weeks and the follow-up phase of 10 weeks of normal diet without consumption of these broccoli sprouts. Anthropometric parameters as body fat mass, body weight, and BMI were determined. Inflammation status was assessed by measuring levels of TNF-α, IL-6, IL-1β and C-reactive protein. IL-6 levels significantly decreased (mean values from 4.76 pg/mL to 2.11 pg/mL with 70 days of broccoli consumption, p<0.001) and during control phase the inflammatory levels were maintained at low grade (mean values from 1.20 pg/mL to 2.66 pg/mL, p<0.001). C-reactive protein significantly decreased as well [279].

An additional potential benefit of sulforaphane is its ability to protect lungs against damage. It is known that the major cause of lethality associated with COVID-19 is acute respiratory distress syndrome (ARDS). It was demonstrated that sulforaphane is effective in the endotoxin model of this condition. In one experiments, BALB/c mice were treated with sulforaphane (50 mg/kg) and 3 days later, ARDS was induced by the administration of LPS (5 mg/kg). The results revealed that sulforaphane significantly decreased lactate dehydrogenase (LDH) activity (as shown by LDH assay), the wet-to-dry ratio of the lungs and the serum levels of interleukin-6 (IL-6) and tumor necrosis factor-α (TNF-α) (measured by ELISA), as well as nuclear factor-κB protein expression in mice with LPS-induced ARDS. Moreover, treatment with sulforaphane significantly inhibited prostaglandin E2 (PGE2) production, and cyclooxygenase-2 (COX-2), matrix metalloproteinase-9 (MMP-9) protein expression (as shown by western blot analysis), as well as inducible nitric oxide synthase (iNOS) activity in mice with LPS-induced ALI. Lastly, the researchers reported pre-treatment with sulforaphane activated the nuclear factor-E2-related factor 2 (Nrf2)/antioxidant response element (ARE) pathway in the mice with LPS-induced ARDS [280].

EGCG is similar to sulforaphane in that it has been reported to possess cancer preventative properties. This compound has been shown to be one of the top therapeutic ingredients in green tea. It is known from epidemiologic studies that green tea consumption associates with chemoprotective effects against cancer [281-291]. In addition, similarly to sulforaphane, EGCG has been shown to inhibit inflammatory mediators. The first suggestion of this were studies shown suppression of the pro-inflammatory transcription factor NF-kappa B. In a detailed molecular study, EGCG, a potent antitumor agent with anti-inflammatory and antioxidant properties was shown to inhibit nitric oxide (NO) generation as a marker of activated macrophages. Inhibition of NO production was observed when cells were cotreated with EGCG and LPS. iNOS activity in soluble extracts of lipopolysaccharide-activated macrophages treated with EGCG (5 and 10 microM) for 6-24 hr was significantly lower than that in macrophages without EGCG treatment. Western blot, reverse transcription-polymerase chain reaction, and Northern blot analyses demonstrated that significantly reduced 130-kDa protein and 4.5-kb mRNA levels of iNOS were expressed in lipopolysaccharide-activated macrophages with EGCG compared with those without EGCG. Electrophoretic mobility shift assay indicated that EGCG blocked the activation of nuclear factor-kappaB, a transcription factor necessary for iNOS induction. EGCG also blocked disappearance of inhibitor kappaB from cytosolic fraction. These results suggest that EGCG decreases the activity and protein levels of iNOS by reducing the expression of iNOS mRNA and the reduction could occur through prevention of the binding of nuclear factor-kappaB to the iNOS promoter [292]. Another study supporting ability of EGCG to suppress NF-kappa B examined a model of atherosclerosis in which exposure of macrophage foam cells to TNF-α results in a downregulation of ABCA1 and a decrease in cholesterol efflux to apoA 1, which is attenuated by pretreatment with EGCG. Moreover, rather than activating the Liver X receptor (LXR) pathway, inhibition of the TNF-α-induced nuclear factor-κB (NF-κB) activity is detected with EGCG treatment in cells. In order to inhibit the NF-κB activity, EGCG can promote the dissociation of the nuclear factor E2-related factor 2 (Nrf2)-Kelch-like ECH-associated protein 1 (Keap1) complex; when the released Nrf2 translocates to the nucleus and activates the transcription of genes containing an ARE element inhibition of NF-κB occurs and Keap1 is separated from the complex to directly interact with IKKβ and thus represses NF-κB function [293].

The anti-inflammatory effects of EGCG can be seen in the ability of this compound to potently inhibit IL-6, the COVID-19 associated cytokine, in a variety of inflammatory settings. For example, in a cardiac infarct model, rats were subjected to myocardial ischemia (30 min) and reperfusion (up to 2 h). Rats were treated with EGCG (10 mg/kg intravenously) or with vehicle at the end of the ischemia period followed by a continuous infusion (EGCG 10 mg/kg/h) during the reperfusion period. In vehicle-treated rats, extensive myocardial injury was associated with tissue neutrophil infiltration as evaluated by myeloperoxidase activity, and elevated levels of plasma creatine phosphokinase. Vehicle-treated rats also demonstrated increased plasma levels of interleukin-6. These events were associated with cytosol degradation of inhibitor kappaB-alpha, activation of IkappaB kinase, phosphorylation of c-Jun, and subsequent activation of nuclear factor-kappaB and activator protein-1 in the infarcted heart. In vivo treatment with EGCG reduced myocardial damage and myeloperoxidase activity. Plasma IL-6 and creatine phosphokinase levels were decreased after EGCG administration. This beneficial effect of EGCG was associated with reduction of nuclear factor-kB and activator protein-1 DNA binding [294]. In an inflammatory model of ulcerative colitis (UC) mice were randomly divided into four groups: Normal control, model (MD), 50 mg/kg/day EGCG treatment and 100 mg/kg/day EGCG treatment. The daily disease activity index (DAI) of the mice was recorded, changes in the organizational structure of the colon were observed and the spleen index (SI) was measured. In addition, levels of interleukin (IL)-6, IL-10, IL-17 and transforming growth factor (TGF)-β1 in the plasma and hypoxia-inducible factor (HIF)-1α and signal transducer and activator of transcription (STAT) 3 protein expression in colon tissues were evaluated. Compared with the MD group, the mice in the two EGCG treatment groups exhibited decreased DAIs and SIs and an attenuation in the colonic tissue erosion. EGCG could reduce the release of IL-6 and IL-17 and regulate the mouse splenic regulatory T-cell (Treg)/T helper 17 cell (Th17) ratio, while increasing the plasma levels of IL-10 and TGF-β1 and decreasing the HIF-1α and STATS protein expression in the colon. The experiments confirmed that EGCG treated mice with experimental colitis by inhibiting the release of IL-6 and regulating the body Treg/Th17 balance [295].

### Example

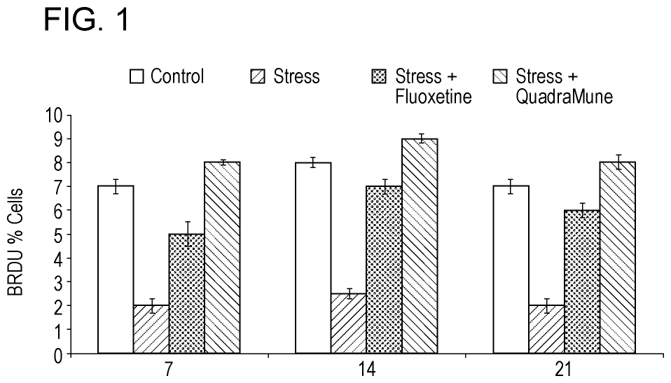

BALB/c mice where exposed to random stress 4 times a day by spinning by the tail for 15 seconds. Controls where not stressed. Stressed mice where treated with Prozac or QuadraMune™ daily. Assessment of endogenous neurogenesis was performed by administering BRDU and assessment of incorporation by histology. Augmented neurogenesis was seen in animals which received Prozac, with enhanced neurogenesis in animals taking QuadraMune™. Results are shown in FIG. 1.

### REFERENCES

1. Hasin, D. S., et al., Epidemiology of Adult DSM-5 Major Depressive Disorder and Its Specifiers in the United States. JAMA Psychiatry, 2018. 75(4): p. 336-346.
2. Penninx, B. W., et al., Inflammatory markers and depressed mood in older persons: results from the Health, Aging and Body Composition study. Biol Psychiatry, 2003. 54(5): p. 566-72.
3. Yoshimura, R., T. Kishi, and N. Iwata, Plasma levels of IL-6 in patients with untreated major depressive disorder: comparison with catecholamine metabolites. Neuropsychiatr Dis Treat, 2019. 15: p. 2655-2661.
4. Xia, Q. R., et al., Increased plasma nesfatin-1 levels may be associated with corticosterone, IL-6, and CRP levels in patients with major depressive disorder. Clin Chim Acta, 2018. 480: p. 107-111.
5. Maes, M., et al., Relationships between interleukin-6 activity, acute phase proteins, and function of the hypothalamic-pituitary-adrenal axis in severe depression. Psychiatry Res, 1993. 49(1): p. 11-27.
6. Maes, M., et al., Relationships between lower plasma L-tryptophan levels and immune-inflammatory variables in depression. Psychiatry Res, 1993. 49(2): p. 151-65.
7. Sluzewska, A., et al., Indicators of immune activation in major depression. Psychiatry Res, 1996. 64(3): p. 161-7.
8. Brambilla, F. and M. Maggioni, Blood levels of cytokines in elderly patients with major depressive disorder. Acta Psychiatr Scand, 1998. 97(4): p. 309-13.
9. Musselman, D. L., et al., Higher than normal plasma interleukin-6 concentrations in cancer patients with depression: preliminary findings. Am J Psychiatry, 2001. 158(8): p. 1252-7.
10. Rief, W., et al., Immunological differences between patients with major depression and somatization syndrome. Psychiatry Res, 2001. 105(3): p. 165-74.
11. Ushiroyama, T., A. Ikeda, and M. Ueki, Elevated plasma interleukin-6 (IL-6) and soluble IL-6 receptor concentrations in menopausal women with and without depression. Int J Gynaecol Obstet, 2002. 79(1): p. 51-2.
12. Glaser, R., et al., Mild depressive symptoms are associated with amplified and prolonged inflammatory responses after influenza virus vaccination in older adults. Arch Gen Psychiatry, 2003. 60(10): p. 1009-14.
13. Lesperance, F., et al., The association between major depression and levels of soluble intercellular adhesion molecule 1, interleukin-6, and C-reactive protein in patients with recent acute coronary syndromes. Am J Psychiatry, 2004. 161(2): p. 271-7.
14. Alesci, S., et al., Major depression is associated with significant diurnal elevations in plasma interleukin-6 levels, a shift of its circadian rhythm, and loss of physiological complexity in its secretion: clinical implications. J Clin Endocrinol Metab, 2005. 90(5): p. 2522-30.
15. Nagata, T., et al., Relationship between plasma concentrations of cytokines, ratio of CD4 and CD8, lymphocyte proliferative responses, and depressive and anxiety state in bulimia nervosa. J Psychosom Res, 2006. 60(1): p. 99-103.
16. Humphreys, D., et al., Interleukin-6 production and deregulation of the hypothalamic-pituitary-adrenal axis in patients with major depressive disorders. Endocrine, 2006. 30(3): p. 371-6.
17. Bremmer, M. A., et al., Inflammatory markers in late-life depression: results from a population-based study. J Affect Disord, 2008. 106(3): p. 249-55.
18. Jacobson, C. M., et al., Depression and IL-6 blood plasma concentrations in advanced cancer patients. Psychosomatics, 2008. 49(1): p. 64-6.
19. Dimopoulos, N., et al., Increased plasma levels of 8-iso-PGF2alpha and IL-6 in an elderly population with depression. Psychiatry Res, 2008. 161(1): p. 59-66.
20. Gabbay, V., et al., Immune system dysregulation in adolescent major depressive disorder. J Affect Disord, 2009. 115(1-2): p. 177-82.
21. Jehn, C. F., et al., Association of IL-6, hypothalamus-pituitary-adrenal axis function, and depression in patients with cancer. Integr Cancer Ther, 2010. 9(3): p. 270-5.
22. Grassi-Oliveira, R., et al., Interleukin-6 and verbal memory in recurrent major depressive disorder. Neuro Endocrinol Lett, 2011. 32(4): p. 540-4.
23. Henje Blom, E., et al., Pro-inflammatory cytokines are elevated in adolescent females with emotional disorders not treated with SSRIs. J Affect Disord, 2012. 136(3): p. 716-23.
24. Hiles, S. A., et al., A meta-analysis of differences in IL-6 and IL-10 between people with and without depression: exploring the causes of heterogeneity. Brain Behav Immun, 2012. 26(7): p. 1180-8.
25. Patas, K., et al., Association between serum brain-derived neurotrophic factor and plasma interleukin-6 in major depressive disorder with melancholic features. Brain Behav Immun, 2014. 36: p. 71-9.
26. Oglodek, E. A., et al., Comparison of chemokines (CCL-5 and SDF-1), chemokine receptors (CCR-5 and CXCR-4) and IL-6 levels in patients with different severities of depression. Pharmacol Rep, 2014. 66(5): p. 920-6.
27. Vogelzangs, N., et al., Cytokine production capacity in depression and anxiety. Transl Psychiatry, 2016. 6(5): p. e825.
28. Cassano, P., et al., Inflammatory cytokines in major depressive disorder: A case-control study. Aust N Z J Psychiatry, 2017. 51(1): p. 23-31.
29. Zheng, T., et al., Increased Dipeptidyl Peptidase-4 Activity Is Associated With High Prevalence of Depression in Middle-Aged and Older Adults: A Cross-Sectional Study. J Clin Psychiatry, 2016. 77(10): p. e1248-e1255.
30. Chen, Y., et al., The Role of Cytokines in the Peripheral Blood of Major Depressive Patients. Clin Lab, 2017. 63(7): p. 1207-1212.
31. Wang, M., et al., The level of IL-6 was associated with sleep disturbances in patients with major depressive disorder. Neuropsychiatr Dis Treat, 2019. 15: p. 1695-1700.
32. Jin, K., et al., Linking peripheral IL-6, IL-1 beta and hypocretin-1 with cognitive impairment from major depression. J Affect Disord, 2020. 277: p. 204-211.
33. Haroon, E., et al., Antidepressant treatment resistance is associated with increased inflammatory markers in patients with major depressive disorder. Psychoneuroendocrinology, 2018. 95: p. 43-49.
34. Kruse, J. L., et al., Inflammation and Improvement of Depression Following Electroconvulsive Therapy in Treatment-Resistant Depression. J Clin Psychiatry, 2018. 79(2).
35. McCormack, D. and D. McFadden, A review of pterostilbene antioxidant activity and disease modification. Oxid Med Cell Longev, 2013. 2013: p. 575482.
36. Paul, B., et al., Occurrence of resveratrol and pterostilbene in age-old darakchasava, an ayurvedic medicine from India. J Ethnopharmacol, 1999. 68(1-3): p. 71-6.
37. Kapetanovic, I. M., et al., Pharmacokinetics, oral bioavailability, and metabolic profile of resveratrol and its dimethylether analog, pterostilbene, in rats. Cancer Chemother Pharmacol, 2011. 68(3): p. 593-601.
38. Perecko, T., et al., Molecular targets of the natural antioxidant pterostilbene: effect on protein kinase C, caspase-3 and apoptosis in human neutrophils in vitro. Neuro Endocrinol Lett, 2010. 31 Suppl 2: p. 84-90.
39. Stivala, L. A., et al., Specific structural determinants are responsible for the antioxidant activity and the cell cycle effects of resveratrol. J Biol Chem, 2001. 276(25): p. 22586-94.
40. Athar, M., et al., Resveratrol: a review of preclinical studies for human cancer prevention. Toxicol Appl Pharmacol, 2007. 224(3): p. 274-83.
41. Bishayee, A., Cancer prevention and treatment with resveratrol: from rodent studies to clinical trials. Cancer Prev Res (Phila), 2009. 2(5): p. 409-18.
42. Hsu, C. L., et al., The inhibitory effect of pterostilbene on inflammatory responses during the interaction of 3T3-L1 adipocytes and RAW 264.7 macrophages. J Agric Food Chem, 2013. 61(3): p. 602-10.
43. McCormack, D., D. McDonald, and D. McFadden, Pterostilbene ameliorates tumor necrosis factor alpha-induced pancreatitis in vitro. J Surg Res, 2012. 178(1): p. 28-32.
44. Erasalo, H., et al., Natural Stilbenoids Have Anti-Inflammatory Properties in Vivo and Down-Regulate the Production of Inflammatory Mediators NO, IL6, and MCP1 Possibly in a PI3K/Akt-Dependent Manner. J Nat Prod, 2018. 81(5): p. 1131-1142.
45. Allijn, I. E., et al., Head-to-Head Comparison of Anti-Inflammatory Performance of Known Natural Products In Vitro. PLoS One, 2016. 11(5): p. e0155325.
46. Meng, X. L., et al., Effects of resveratrol and its derivatives on lipopolysaccharide-induced microglial activation and their structure-activity relationships. Chem Biol Interact, 2008. 174(1): p. 51-9.
47. Zhang, L., et al., Pterostilbene protects vascular endothelial cells against oxidized low-density lipoprotein-induced apoptosis in vitro and in vivo. Apoptosis, 2012. 17(1): p. 25-36.
48. Park, S. H., et al., Pterostilbene, an Active Constituent of Blueberries, Stimulates Nitric Oxide Production via Activation of Endothelial Nitric Oxide Synthase in Human Umbilical Vein Endothelial Cells. Plant Foods Hum Nutr, 2015. 70(3): p. 263-8.
49. Chen, Z. W., et al., Pterostilbene protects against uraemia serum-induced endothelial cell damage via activation of Keap1/Nrf2/HO-1 signaling. Int Urol Nephrol, 2018. 50(3): p. 559-570.
50. Chen, C., et al., Effect of resveratrol combined with atorvastatin on re-endothelialization after drug-eluting stents implantation and the underlying mechanism. Life Sci, 2020. 245: p. 117349.
51. Bekpinar, S., et al., Resveratrol ameliorates the cyclosporine-induced vascular and renal impairments: possible impact of the modulation of renin-angiotensin system. Can J Physiol Pharmacol, 2019. 97(12): p. 1115-1123.
52. Toama, M. A., T. S. El-Alfy, and H. M. El-Fatatry, Antimicrobial activity of the volatile oil of Nigella sativa Linneaus seeds. Antimicrob Agents Chemother, 1974. 6(2): p. 225-6.
53. Padhye, S., et al., From here to eternity—the secret of Pharaohs: Therapeutic potential of black cumin seeds and beyond. Cancer Ther, 2008. 6(b): p. 495-510.
54. Toppozada, H. H., H. A. Mazloum, and M. el-Dakhakhny, The antibacterial properties of the Nigella sativa l. seeds. Active principle with some clinical applications. J Egypt Med Assoc, 1965. 48: p. Suppl: 187-202.
55. Agarwal, R., M. D. Kharya, and R. Shrivastava, Antimicrobial & anthelmintic activities of the essential oil of Nigella sativa Linn. Indian J Exp Biol, 1979. 17(11): p. 1264-5.
56. Hanafy, M. S. and M. E. Hatem, Studies on the antimicrobial activity of Nigella sativa seed (black cumin). J Ethnopharmacol, 1991. 34(2-3): p. 275-8.
57. Morsi, N. M., Antimicrobial effect of crude extracts of Nigella sativa on multiple antibiotics-resistant bacteria. Acta Microbiol Pol, 2000. 49(1): p. 63-74.
58. Randhawa, M. A., et al., An active principle of Nigella sativa L., thymoquinone, showing significant antimicrobial activity against anaerobic bacteria. J Intercult Ethnopharmacol, 2017. 6(1): p. 97-101.
59. Hashem-Dabaghian, F., et al., Combination of Nigella sativa and Honey in Eradication of Gastric Helicobacter pylori Infection. Iran Red Crescent Med J, 2016. 18(11): p. e23771.
60. Mouwakeh, A., et al., Nigella sativa essential oil and its bioactive compounds as resistance modifiers against Staphylococcus aureus. Phytother Res, 2019. 33(4): p. 1010-1018.
61. Salem, M. L. and M. S. Hossain, Protective effect of black seed oil from Nigella sativa against murine cytomegalovirus infection. Int J Immunopharmacol, 2000. 22(9): p. 729-40.
62. Barakat, E. M., L. M. El Wakeel, and R. S. Hagag, Effects of Nigella sativa on outcome of hepatitis C in Egypt. World J Gastroenterol, 2013. 19(16): p. 2529-36.
63. Onifade, A. A., A. P. Jewell, and W. A. Adedeji, Nigella sativa concoction induced sustained seroreversion in HIV patient. Afr J Tradit Complement Altern Med, 2013. 10(5): p. 332-5.
64. Oyero, O. G., et al., Selective Inhibition of Hepatitis C Virus Replication by Alpha-Zam, a Nigella Sativa Seed Formulation. Afr J Tradit Complement Altern Med, 2016. 13(6): p. 144-148.
65. Islam, S. K., et al., Antifungal activities of the oils of Nigella sativa seeds. Pak J Pharm Sci, 1989. 2(1): p. 25-8.
66. Rogozhin, E. A., et al., Novel antifungal defensins from Nigella sativa L. seeds. Plant Physiol Biochem, 2011. 49(2): p. 131-7.
67. El Gazzar, M., et al., Downregulation of leukotriene biosynthesis by thymoquinone attenuates airway inflammation in a mouse model of allergic asthma. Biochim Biophys Acta, 2006. 1760(7): p. 1088-95.
68. El Gazzar, M., et al., Anti-inflammatory effect of thymoquinone in a mouse model of allergic lung inflammation. Int Immunopharmacol, 2006. 6(7): p. 1135-42.
69. Abbas, A. T., et al., Effect of dexamethasone and Nigella sativa on peripheral blood eosinophil count, IgG1 and IgG2a, cytokine profiles and lung inflammation in murine model of allergic asthma. Egypt J Immunol, 2005. 12(1): p. 95-102.
70. Boskabady, M. H., N. Mohsenpoor, and L. Takaloo, Antiasthmatic effect of Nigella sativa in airways of asthmatic patients. Phytomedicine, 2010. 17(10): p. 707-13.
71. Tayman, C., et al., Protective Effects of Nigella sativa Oil in Hyperoxia-Induced Lung Injury. Arch Bronconeumol, 2013. 49(1): p. 15-21.
72. Balaha, M. F., et al., Oral Nigella sativa oil ameliorates ovalbumin-induced bronchial asthma in mice. Int Immunopharmacol, 2012. 14(2): p. 224-31.
73. Keyhanmanesh, R., et al., The effect of single dose of thymoquinone, the main constituents of Nigella sativa, in guinea pig model of asthma. Bioimpacts, 2014. 4(2): p. 75-81.
74. Ali, B. H., et al., The effect of thymoquinone treatment on the combined renal and pulmonary toxicity of cisplatin and diesel exhaust particles. Exp Biol Med (Maywood), 2015. 240(12): p. 1698-707.
75. Keyhanmanesh, R., et al., The Protective Effect of alpha-Hederin, the Active Constituent of Nigella sativa, on Lung Inflammation and Blood Cytokines in Ovalbumin Sensitized Guinea Pigs. Phytother Res, 2015. 29(11): p. 1761-7.
76. Pourgholamhossein, F., et al., Thymoquinone effectively alleviates lung fibrosis induced by paraquat herbicide through down-regulation of pro-fibrotic genes and inhibition of oxidative stress. Environ Toxicol Pharmacol, 2016. 45: p. 340-5.
77. Koshak, A., et al., Nigella sativa Supplementation Improves Asthma Control and Biomarkers: A Randomized, Double-Blind, Placebo-Controlled Trial. Phytother Res, 2017. 31(3): p. 403-409.
78. Salem, A. M., et al., Effect of Nigella sativa supplementation on lung function and inflammatory mediatorsin partly controlled asthma: a randomized controlled trial. Ann Saudi Med, 2017. 37(1): p. 64-71.
79. Abidi, A., et al., Nigella sativa, a traditional Tunisian herbal medicine, attenuates bleomycin-induced pulmonary fibrosis in a rat model. Biomed Pharmacother, 2017. 90: p. 626-637.
80. Poursalehi, H. R., et al., Early and late preventive effect of Nigella sativa on the bleomycin-induced pulmonary fibrosis in rats: An experimental study. Avicenna J Phytomed, 2018. 8(3): p. 263-275.
81. Ikhsan, M., et al., Nigella sativa as an anti-inflammatory agent in asthma. BMC Res Notes, 2018. 11(1): p. 744.
82. Burits, M. and F. Bucar, Antioxidant activity of Nigella sativa essential oil. Phytother Res, 2000. 14(5): p. 323-8.
83. Badary, O. A., et al., Thymoquinone is a potent superoxide anion scavenger. Drug Chem Toxicol, 2003. 26(2): p. 87-98.
84. Ismail, M., G. Al-Naqeep, and K. W. Chan, Nigella sativa thymoquinone-rich fraction greatly improves plasma antioxidant capacity and expression of antioxidant genes in hypercholesterolemic rats. Free Radic Biol Med, 2010. 48(5): p. 664-72.
85. Awan, M. A., et al., Antioxidant activity of Nigella sativa Seeds Aqueous Extract and its use for cryopreservation of buffalo spermatozoa. Andrologia, 2018. 50(6): p. e13020.
86. Ardiana, M., et al., Effect of Nigella sativa Supplementation on Oxidative Stress and Antioxidant Parameters: A Meta-Analysis of Randomized Controlled Trials. Scientific World Journal, 2020. 2020: p. 2390706.
87. Al-Awadi, F. M. and K. A. Gumaa, Studies on the activity of individual plants of an antidiabetic plant mixture. Acta Diabetol Lat, 1987. 24(1): p. 37-41.
88. Meral, I., et al., Effect of Nigella sativa on glucose concentration, lipid peroxidation, anti-oxidant defence system and liver damage in experimentally-induced diabetic rabbits. J Vet Med A Physiol Pathol Clin Med, 2001. 48(10): p. 593-9.
89. El-Dakhakhny, M., et al., The hypoglycemic effect of Nigella sativa oil is mediated by extrapancreatic actions. Planta Med, 2002. 68(5): p. 465-6.
90. Kanter, M., et al., Partial regeneration/proliferation of the beta-cells in the islets of Langerhans by Nigella sativa L. in streptozotocin-induced diabetic rats. Tohoku J Exp Med, 2003. 201(4): p. 213-9.
91. Fararh, K. M., et al., Mechanisms of the hypoglycaemic and immunopotentiating effects of Nigella sativa L. oil in streptozotocin-induced diabetic hamsters. Res Vet Sci, 2004. 77(2): p. 123-9.
92. Kanter, M., et al., Effects of Nigella sativa on oxidative stress and beta-cell damage in streptozotocin-induced diabetic rats. Anat Rec A Discov Mol Cell Evol Biol, 2004. 279(1): p. 685-91.
93. Le, P. M., et al., The petroleum ether extract of Nigella sativa exerts lipid-lowering and insulin-sensitizing actions in the rat. J Ethnopharmacol, 2004. 94(2-3): p. 251-9.
94. Rchid, H., et al., Nigella sativa seed extracts enhance glucose-induced insulin release from rat-isolated Langerhans islets. Fundam Clin Pharmacol, 2004. 18(5): p. 525-9.
95. Bamosa, A. O., et al., Effect of Nigella sativa seeds on the glycemic control of patients with type 2 diabetes mellitus. Indian J Physiol Pharmacol, 2010. 54(4): p. 344-54.
96. Badar, A., et al., Effect of Nigella sativa supplementation over a one-year period on lipid levels, blood pressure and heart rate in type-2 diabetic patients receiving oral hypoglycemic agents: nonrandomized clinical trial. Ann Saudi Med, 2017. 37(1): p. 56-63.
97. Abdelrazek, H. M. A., et al., Black Seed Thymoquinone Improved Insulin Secretion, Hepatic Glycogen Storage, and Oxidative Stress in Streptozotocin-Induced Diabetic Male Wistar Rats. Oxid Med Cell Longev, 2018. 2018: p. 8104165.
98. Salomi, M. J., et al., Anti-cancer activity of Nigella sativa. Anc Sci Life, 1989. 8(3-4): p. 262-6.
99. Salomi, M. J., S. C. Nair, and K R Panikkar, Inhibitory effects of Nigella sativa and saffron (Crocus sativus) on chemical carcinogenesis in mice. Nutr Cancer, 1991. 16(1): p. 67-72.
100. Salomi, N.J., et al., Antitumour principles from Nigella sativa seeds. Cancer Lett, 1992. 63(1): p. 41-6.
101. Worthen, D. R., O. A. Ghosheh, and P. A. Crooks, The in vitro anti-tumor activity of some crude and purified components of blackseed, Nigella sativa L. Anticancer Res, 1998. 18(3A): p. 1527-32.
102. Badary, O. A., Thymoquinone attenuates ifosfamide-induced Fanconi syndrome in rats and enhances its antitumor activity in mice. J Ethnopharmacol, 1999. 67(2): p. 135-42.
103. Kumara, S. S. and B. T. Huat, Extraction, isolation and characterisation of antitumor principle, alpha-hederin, from the seeds of Nigella sativa. Planta Med, 2001. 67(1): p. 29-32.
104. Gali-Muhtasib, H., et al., Thymoquinone extracted from black seed triggers apoptotic cell death in human colorectal cancer cells via a p53-dependent mechanism. Int J Oncol, 2004. 25(4): p. 857-66.
105. Yi, T., et al., Thymoquinone inhibits tumor angiogenesis and tumor growth through suppressing AKT and extracellular signal-regulated kinase signaling pathways. Mol Cancer Ther, 2008. 7(7): p. 1789-96.
106. Banerjee, S., et al., Antitumor activity of gemcitabine and oxaliplatin is augmented by thymoquinone in pancreatic cancer. Cancer Res, 2009. 69(13): p. 5575-83.
107. Baharetha, H. M., et al., Proapoptotic and antimetastatic properties of supercritical CO2 extract of Nigella sativa Linn. against breast cancer cells. J Med Food, 2013. 16(12): p. 1121-30.
108. Al-Sheddi, E. S., et al., Cytotoxicity of Nigella sativa seed oil and extract against human lung cancer cell line. Asian Pac J Cancer Prev, 2014. 15(2): p. 983-7.
109. Linjawi, S. A., et al., Evaluation of the protective effect of Nigella sativa extract and its primary active component thymoquinone against DMBA-induced breast cancer in female rats. Arch Med Sci, 2015. 11(1): p. 220-9.
110. Khalife, R., et al., Thymoquinone from Nigella sativa Seeds Promotes the Antitumor Activity of Noncytotoxic Doses of Topotecan in Human Colorectal Cancer Cells in Vitro. Planta Med, 2016. 82(4): p. 312-21.
111. Relles, D., et al., Thymoquinone Promotes Pancreatic Cancer Cell Death and Reduction of Tumor Size through Combined Inhibition of Histone Deacetylation and Induction of Histone Acetylation. Adv Prev Med, 2016. 2016: p. 1407840.
112. Arumugam, P., et al., Thymoquinone inhibits the migration of mouse neuroblastoma (Neuro-2a) cells by down-regulating MMP-2 and MMP-9. Chin J Nat Med, 2016. 14(12): p. 904-912.
113. Kabil, N., et al., Thymoquinone inhibits cell proliferation, migration, and invasion by regulating the elongation factor 2 kinase (eEF-2K) signaling axis in triple-negative breast cancer. Breast Cancer Res Treat, 2018. 171(3): p. 593-605.
114. Daba, M. H. and M. S. Abdel-Rahman, Hepatoprotective activity of thymoquinone in isolated rat hepatocytes. Toxicol Lett, 1998. 95(1): p. 23-9.
115. Nagi, M. N., et al., Thymoquinone protects against carbon tetrachloride hepatotoxicity in mice via an antioxidant mechanism. Biochem Mol Biol Int, 1999. 47(1): p. 153-9.
116. el-Dakhakhny, M., N. I. Mady, and M. A. Halim, Nigella sativa L. oil protects against induced hepatotoxicity and improves serum lipid profile in rats. Arzneimittelforschung, 2000. 50(9): p. 832-6.
117. Turkdogan, M. K., et al., The role of antioxidant vitamins (C and E), selenium and Nigella sativa in the prevention of liver fibrosis and cirrhosis in rabbits: new hopes. Dtsch Tierarztl Wochenschr, 2001. 108(2): p. 71-3.
118. Mahmoud, M. R., H. S. El-Abhar, and S. Saleh, The effect of Nigella sativa oil against the liver damage induced by Schistosoma mansoni infection in mice. J Ethnopharmacol, 2002. 79(1): p. 1-11.
119. Mansour, M. A., et al., Effects of volatile oil constituents of Nigella sativa on carbon tetrachloride-induced hepatotoxicity in mice: evidence for antioxidant effects of thymoquinone. Res Commun Mol Pathol Pharmacol, 2001. 110(3-4): p. 239-51.
120. Al-Ghamdi, M. S., Protective effect of Nigella sativa seeds against carbon tetrachloride-induced liver damage. Am J Chin Med, 2003. 31(5): p. 721-8.
121. Farooqui, Z., et al., Oral administration of Nigella sativa oil ameliorates the effect of cisplatin on membrane enzymes, carbohydrate metabolism and oxidative damage in rat liver. Toxicol Rep, 2016. 3: p. 328-335.
122. Mabrouk, A., et al., Protective effect of thymoquinone against lead-induced hepatic toxicity in rats. Environ Sci Pollut Res Int, 2016. 23(12): p. 12206-15.
123. Adam, G. O., et al., Hepatoprotective effects of Nigella sativa seed extract against acetaminophen-induced oxidative stress. Asian Pac J Trop Med, 2016. 9(3): p. 221-7.
124. El-Far, A. H., et al., Hepatoprotective efficacy of Nigella sativa seeds dietary supplementation against lead acetate-induced oxidative damage in rabbit—Purification and characterization of glutathione peroxidase. Biomed Pharmacother, 2017. 89: p. 711-718.
125. Tuorkey, M. J., Therapeutic Potential of Nigella sativa Oil Against Cyclophosphamide-Induced DNA Damage and Hepatotoxicity. Nutr Cancer, 2017. 69(3): p. 498-504.
126. Noorbakhsh, M. F., et al., An Overview of Hepatoprotective Effects of Thymoquinone. Recent Pat Food Nutr Agric, 2018. 9(1): p. 14-22.
127. Bouhlel, A., et al., Thymoquinone protects rat liver after partial hepatectomy under ischaemia/reperfusion through oxidative stress and endoplasmic reticulum stress prevention. Clin Exp Pharmacol Physiol, 2018.
128. Ebru, U., et al., Cardioprotective effects of Nigella sativa oil on cyclosporine A-induced cardiotoxicity in rats. Basic Clin Pharmacol Toxicol, 2008. 103(6): p. 574-80.
129. Yar, T., et al., Effects of Nigella sativa supplementation for one month on cardiac reserve in rats. Indian J Physiol Pharmacol, 2008. 52(2): p. 141-8.
130. Seif, A. A., Nigella sativa attenuates myocardial ischemic reperfusion injury in rats. J Physiol Biochem, 2013. 69(4): p. 937-44.
131. Randhawa, M. A., M. S. Alghamdi, and S. K. Maulik, The effect of thymoquinone, an active component of Nigella sativa, on isoproterenol induced myocardial injury. Pak J Pharm Sci, 2013. 26(6): p. 1215-9.
132. Bamosa, A., et al., Nigella sativa: A potential natural protective agent against cardiac dysfunction in patients with type 2 diabetes mellitus. J Family Community Med, 2015. 22(2): p. 88-95.
133. Ojha, S., et al., Thymoquinone Protects against Myocardial Ischemic Injury by Mitigating Oxidative Stress and Inflammation. Evid Based Complement Alternat Med, 2015. 2015: p. 143629.
134. Gonca, E. and C. Kurt, Cardioprotective effect of Thymoquinone: A constituent of Nigella sativa L., against myocardial ischemia/reperfusion injury and ventricular arrhythmias in anaesthetized rats. Pak J Pharm Sci, 2015. 28(4): p. 1267-73.
135. Hebi, M., et al., Cardiovascular effect of Nigella sativa L. Aqueous Extract in Normal Rats. Cardiovasc Hematol Disord Drug Targets, 2016. 16(1): p. 47-55.
136. Norouzi, F., et al., The effect of Nigella sativa on inflammation-induced myocardial fibrosis in male rats. Res Pharm Sci, 2017. 12(1): p. 74-81.
137. Al Asoom, L. I., Coronary angiogenic effect of long-term administration of Nigella sativa. BMC Complement Altern Med, 2017. 17(1): p. 308.
138. Danaei, G. H., et al., Protective effect of thymoquinone, the main component of Nigella sativa, against diazinon cardio-toxicity in rats. Drug Chem Toxicol, 2019. 42(6): p. 585-591.
139. Lu, Y., et al., Thymoquinone Attenuates Myocardial Ischemia/Reperfusion Injury Through Activation of SIRT1 Signaling. Cell Physiol Biochem, 2018. 47(3): p. 1193-1206.
140. Xiao, J., et al., The cardioprotective effect of thymoquinone on ischemia-reperfusion injury in isolated rat heart via regulation of apoptosis and autophagy. J Cell Biochem, 2018. 119(9): p. 7212-7217.
141. Altun, E., et al., Protective Effect of Nigella sativa Oil on Myocardium in Streptozotocin-Induced Diabetic Rats. Acta Endocrinol (Buchar), 2019. 15(3): p. 289-294.
142. Razmpoosh, E., et al., The effect of Nigella sativa supplementation on cardiovascular risk factors in obese and overweight women: a crossover, double-blind, placebo-controlled randomized clinical trial. Eur J Nutr, 2020.
143. Radad, K., et al., Thymoquinone ameliorates lead-induced brain damage in Sprague Dawley rats. Exp Toxicol Pathol, 2014. 66(1): p. 13-7.
144. Ismail, N., et al., Thymoquinone prevents beta-amyloid neurotoxicity in primary cultured cerebellar granule neurons. Cell Mol Neurobiol, 2013. 33(8): p. 1159-69.
145. Hobbenaghi, R., et al., Neuroprotective effects of Nigella sativa extract on cell death in hippocampal neurons following experimental global cerebral ischemia-reperfusion injury in rats. J Neurol Sci, 2014. 337(1-2): p. 74-9.
146. Hosseini, M., et al., Effects of the hydro-alcoholic extract of Nigella sativa on scopolamine-induced spatial memory impairment in rats and its possible mechanism. Chin J Integr Med, 2015. 21(6): p. 438-44.
147. Akhtar, M., et al., Neuroprotective study of Nigella sativa-loaded oral provesicular lipid formulation: in vitro and ex vivo study. Drug Deliv, 2014. 21(6): p. 487-94.
148. Sedaghat, R., M. Roghani, and M. Khalili, Neuroprotective effect of thymoquinone, the Nigella sativa bioactive compound, in 6-hydroxydopamine-induced hemi-parkinsonian rat model. Iran J Pharm Res, 2014. 13(1): p. 227-34.
149. Vafaee, F., et al., The Effects of Nigella sativa Hydro-alcoholic Extract on Memory and Brain Tissues Oxidative Damage after Repeated Seizures in Rats. Iran J Pharm Res, 2015. 14(2): p. 547-57.
150. Islam, M. H., I. Z. Ahmad, and M. T. Salman, Neuroprotective effects of Nigella sativa extracts during germination on central nervous system. Pharmacogn Mag, 2015. 11(Suppl 1): p. S182-9.
151. Noor, N. A., et al., Nigella sativa amliorates inflammation and demyelination in the experimental autoimmune encephalomyelitis-induced Wistar rats. Int J Clin Exp Pathol, 2015. 8(6): p. 6269-86.
152. Abbasnezhad, A., et al., The effects of hydroalcoholic extract of Nigella sativa seed on oxidative stress in hippocampus of STZ-induced diabetic rats. Avicenna J Phytomed, 2015. 5(4): p. 333-40.
153. Khazdair, M. R., The Protective Effects of Nigella sativa and Its Constituents on Induced Neurotoxicity. J Toxicol, 2015. 2015: p. 841823.
154. Gokce, E. C., et al., Neuroprotective effects of thymoquinone against spinal cord ischemia-reperfusion injury by attenuation of inflammation, oxidative stress, and apoptosis. J Neurosurg Spine, 2016. 24(6): p. 949-59.
155. Sahak, M. K., et al., The Role of Nigella sativa and Its Active Constituents in Learning and Memory. Evid Based Complement Alternat Med, 2016. 2016: p. 6075679.
156. Hosseinzadeh, H., et al., Attenuation of morphine tolerance and dependence by thymoquinone in mice. Avicenna J Phytomed, 2016. 6(1): p. 55-66.
157. Norouzi, F., et al., The effects of Nigella sativa on sickness behavior induced by lipopolysaccharide in male Wistar rats. Avicenna J Phytomed, 2016. 6(1): p. 104-16.
158. Malik, T., et al., Nigella sativa Oil Reduces Extrapyramidal Symptoms (EPS)-Like Behavior in Haloperidol-Treated Rats. Neurochem Res, 2016. 41(12): p. 3386-3398.
159. Farooqui, Z., et al., Protective effect of Nigella sativa oil on cisplatin induced nephrotoxicity and oxidative damage in rat kidney. Biomed Pharmacother, 2017. 85: p. 7-15.
160. Soleimannejad, K., et al., Effects of Nigella sativa Extract on Markers of Cerebral Angiogenesis after Global Ischemia of Brain in Rats. J Stroke Cerebrovasc Dis, 2017. 26(7): p. 1514-1520.
161. Shao, Y. Y., et al., Thymoquinone Attenuates Brain Injury via an Anti-oxidative Pathway in a Status Epilepticus Rat Model. Transl Neurosci, 2017. 8: p. 9-14.
162. Asiaei, F., et al., Neuroprotective effects of Nigella sativa extract upon the hippocampus in PTU-induced hypothyroidism juvenile rats: A stereological study. Metab Brain Dis, 2017. 32(5): p. 1755-1765.
163. Razin, M. A. F., et al., Immune responses to killed reassorted influenza virus supplemented with natural adjuvants. Acta Microbiol Immunol Hung, 2017. 64(3): p. 313-330.
164. Abulfadl, Y. S., et al., Thymoquinone alleviates the experimentally induced Alzheimer's disease inflammation by modulation of TLRs signaling. Hum Exp Toxicol, 2018. 37(10): p. 1092-1104.
165. Abulfadl, Y. S., et al., Protective effects of thymoquinone on D-galactose and aluminum chloride induced neurotoxicity in rats: biochemical, histological and behavioral changes. Neurol Res, 2018. 40(4): p. 324-333.
166. Cascella, M., et al., Dissecting the Potential Roles of Nigella sativa and Its Constituent Thymoquinone on the Prevention and on the Progression of Alzheimer's Disease. Front Aging Neurosci, 2018. 10: p. 16.
167. Norouzi, F., et al., Memory enhancing effect of Nigella Sativa hydro-alcoholic extract on lipopolysaccharide-induced memory impairment in rats. Drug Chem Toxicol, 2019. 42(3): p. 270-279.
168. Fouad, I. A., et al., Neuromodulatory Effect of Thymoquinone in Attenuating Glutamate-Mediated Neurotoxicity Targeting the Amyloidogenic and Apoptotic Pathways. Front Neurol, 2018. 9: p. 236.
169. Firdaus, F., et al., Thymoquinone alleviates arsenic induced hippocampal toxicity and mitochondrial dysfunction by modulating mPTP in Wistar rats. Biomed Pharmacother, 2018. 102: p. 1152-1160.
170. Anaeigoudari, A., et al., Protective effects of Nigella sativa on synaptic plasticity impairment induced by lipopolysaccharide. Vet Res Forum, 2018. 9(1): p. 27-33.
171. Cobourne-Duval, M. K., et al., Thymoquinone increases the expression of neuroprotective proteins while decreasing the expression of pro-inflammatory cytokines and the gene expression NFkappaB pathway signaling targets in LPS/IFNgamma-activated BV-2 microglia cells. J Neuroimmunol, 2018. 320: p. 87-97.
172. Poorgholam, P., P. Yaghmaei, and Z. Hajebrahimi, Thymoquinone recovers learning function in a rat model of Alzheimer's disease. Avicenna J Phytomed, 2018. 8(3): p. 188-197.
173. Firdaus, F., et al., Anxiolytic and anti-inflammatory role of thymoquinone in arsenic-induced hippocampal toxicity in Wistar rats. Heliyon, 2018. 4(6): p. e00650.
174. Ustun, R., et al., Thymoquinone protects DRG neurons from axotomy-induced cell death. Neurol Res, 2018. 40(11): p. 930-937.
175. Butt, U. J., et al., Protective effects of Nigella sativa L. seed extract on lead induced neurotoxicity during development and early life in mouse models. Toxicol Res (Camb), 2018. 7(1): p. 32-40.
176. Ustun, R., et al., Thymoquinone prevents cisplatin neurotoxicity in primary DRG neurons. Neurotoxicology, 2018. 69: p. 68-76.
177. Alhibshi, A.H., A. Odawara, and I. Suzuki, Neuroprotective efficacy of thymoquinone against amyloid beta-induced neurotoxicity in human induced pluripotent stem cell-derived cholinergic neurons. Biochem Biophys Rep, 2019. 17: p. 122-126.
178. Fanoudi, S., et al., Nigella sativa and thymoquinone attenuate oxidative stress and cognitive impairment following cerebral hypoperfusion in rats. Metab Brain Dis, 2019. 34(4): p. 1001-1010.
179. Hamdan, A. M., et al., Thymoquinone therapy remediates elevated brain tissue inflammatory mediators induced by chronic administration of food preservatives. Sci Rep, 2019. 9(1): p. 7026.
180. Nazir, S., et al., Thymoquinone harbors protection against Concanavalin A-induced behavior deficit in BALB/c mice model. J Food Biochem, 2020: p. e13348.
181. Omran, O. M., Effects of thymoquinone on STZ-induced diabetic nephropathy: an immunohistochemical study. Ultrastruct Pathol, 2014. 38(1): p. 26-33.
182. Mousavi, G. and D. Mohajeri, Effect of ground black seeds (Nigella sativa L.) on renal tubular cell apoptosis induced by ischemia/reperfusion injury in the rats. Iran J Basic Med Sci, 2014. 17(12): p. 1032-5.
183. Al-Gayyar, M. M., et al., Nigella sativa oil attenuates chronic nephrotoxicity induced by oral sodium nitrite: Effects on tissue fibrosis and apoptosis. Redox Rep, 2016. 21(2): p. 50-60.
184. Erboga, M., et al., Thymoquinone Ameliorates Cadmium-Induced Nephrotoxicity, Apoptosis, and Oxidative Stress in Rats is Based on its Anti-Apoptotic and Anti-Oxidant Properties. Biol Trace Elem Res, 2016. 170(1): p. 165-72.
185. Mousavi, G., Study on the effect of black cumin (Nigella sativa Linn.) on experimental renal ischemia-reperfusion injury in rats. Acta Cir Bras, 2015. 30(8): p. 542-50.
186. Canayakin, D., et al., Paracetamol-induced nephrotoxicity and oxidative stress in rats: the protective role of Nigella sativa. Pharm Biol, 2016. 54(10): p. 2082-91.
187. Caskurlu, T., et al., Protective Effect of Nigella sativa on Renal Reperfusion Injury in Rat. Iran J Kidney Dis, 2016. 10(3): p. 135-43.
188. Hosseinian, S., et al., The protective effect of Nigella sativa against cisplatin-induced nephrotoxicity in rats. Avicenna J Phytomed, 2016. 6(1): p. 44-54.
189. Hammad, F. T. and L. Lubbad, The effect of thymoquinone on the renal functions following ischemia-reperfusion injury in the rat. Int J Physiol Pathophysiol Pharmacol, 2016. 8(4): p. 152-159.
190. Ahmed, J. H. and I. M. Abdulmajeed, Effect of Nigella sativa (black seeds) against methotrexate-induced nephrotoxicity in mice. J Intercult Ethnopharmacol, 2017. 6(1): p. 9-13.
191. Hosseinian, S., et al., Renoprotective effect of Nigella sativa against cisplatin-induced nephrotoxicity and oxidative stress in rat. Saudi J Kidney Dis Transpl, 2018. 29(1): p. 19-29.
192. Shaterzadeh-Yazdi, H., et al., An Overview on Renoprotective Effects of Thymoquinone. Kidney Dis (Basel), 2018. 4(2): p. 74-82.
193. Alsuhaibani, A. M. A., Effect of Nigella sativa against cisplatin induced nephrotoxicity in rats. Ital J Food Saf, 2018. 7(2): p. 7242.
194. Alam, M. A., et al., Evaluation of safety and efficacy profile of Nigella sativa oil as an add-on therapy, in addition to alpha-keto analogue of essential amino acids in patients with chronic kidney disease. Saudi J Kidney Dis Transpl, 2020. 31(1): p. 21-31.
195. Enomoto, S., et al., Hematological studies on black cumin oil from the seeds of Nigella sativa L. Biol Pharm Bull, 2001. 24(3): p. 307-10.
196. Zaoui, A., et al., Effects of Nigella sativa fixed oil on blood homeostasis in rat. J Ethnopharmacol, 2002. 79(1): p. 23-6.
197. Alici, O., et al., Treatment of Nigella sativa in experimental sepsis model in rats. Pak J Pharm Sci, 2011. 24(2): p. 227-31.
198. Bayir, Y., et al., Nigella sativa as a potential therapy for the treatment of lung injury caused by cecal ligation and puncture-induced sepsis model in rats. Cell Mol Biol (Noisy-le-grand), 2012. 58 Suppl: p. OL1680-7.
199. Guo, L. P., et al., Effect of Thymoquinone on Acute Kidney Injury Induced by Sepsis in BALB/c Mice. Biomed Res Int, 2020. 2020: p. 1594726.
200. Idris-Khodja, N. and V. Schini-Kerth, Thymoquinone improves aging-related endothelial dysfunction in the rat mesenteric artery. Naunyn Schmiedebergs Arch Pharmacol, 2012. 385(7): p. 749-58.
201. Ahmad, A., R. M. Khan, and K. M. Alkharfy, Effects of selected bioactive natural products on the vascular endothelium. J Cardiovasc Pharmacol, 2013. 62(2): p. 111-21.
202. Cuce, G., et al., Effects of Nigella sativa L. seed oil on intima-media thickness and Bax and Caspase 3 expression in diabetic rat aorta. Anatol J Cardiol, 2016. 16(7): p. 460-466.
203. Abbasnezhad, A., et al., Nigella sativa seed decreases endothelial dysfunction in streptozotocin-induced diabetic rat aorta. Avicenna J Phytomed, 2016. 6(1): p. 67-76.
204. Guan, D., et al., Thymoquinone protects against cerebral small vessel disease: Role of antioxidant and anti-inflammatory activities. J Biol Regul Homeost Agents, 2018. 32(2): p. 225-231.
205. Chakravarty, N., Inhibition of histamine release from mast cells by nigellone. Ann Allergy, 1993. 70(3): p. 237-42.
206. Houghton, P. J., et al., Fixed oil of Nigella sativa and derived thymoquinone inhibit eicosanoid generation in leukocytes and membrane lipid peroxidation. Planta Med, 1995. 61(1): p. 33-6.
207. Al-Ghamdi, M. S., The anti-inflammatory, analgesic and antipyretic activity of Nigella sativa. J Ethnopharmacol, 2001. 76(1): p. 45-8.
208. El-Dakhakhny, M., et al., Nigella sativa oil, nigellone and derived thymoquinone inhibit synthesis of 5-lipoxygenase products in polymorphonuclear leukocytes from rats. J Ethnopharmacol, 2002. 81(2): p. 161-4.
209. Mansour, M. and S. Tornhamre, Inhibition of 5-lipoxygenase and leukotriene C4 synthase in human blood cells by thymoquinone. J Enzyme Inhib Med Chem, 2004. 19(5): p. 431-6.
210. Marsik, P., et al., In vitro inhibitory effects of thymol and quinones of Nigella sativa seeds on cyclooxygenase-1- and -2-catalyzed prostaglandin E2 biosyntheses. Planta Med, 2005. 71(8): p. 739-42.
211. Kanter, M., O. Coskun, and H. Uysal, The antioxidative and antihistaminic effect of Nigella sativa and its major constituent, thymoquinone on ethanol-induced gastric mucosal damage. Arch Toxicol, 2006. 80(4): p. 217-24.
212. Sethi, G., K. S. Ahn, and B. B. Aggarwal, Targeting nuclear factor-kappa B activation pathway by thymoquinone: role in suppression of antiapoptotic gene products and enhancement of apoptosis. Mol Cancer Res, 2008. 6(6): p. 1059-70.
213. Mat, M. C., A. S. Mohamed, and S. S. Hamid, Primary human monocyte differentiation regulated by Nigella sativa pressed oil. Lipids Health Dis, 2011. 10: p. 216.
214. Umar, S., et al., Thymoquinone inhibits TNF-alpha-induced inflammation and cell adhesion in rheumatoid arthritis synovial fibroblasts by ASK1 regulation. Toxicol Appl Pharmacol, 2015. 287(3): p. 299-305.
215. Hadi, V., et al., Effects of Nigella sativa oil extract on inflammatory cytokine response and oxidative stress status in patients with rheumatoid arthritis: a randomized, double-blind, placebo-controlled clinical trial. Avicenna J Phytomed, 2016. 6(1): p. 34-43.
216. Hossen, M. J., et al., Thymoquinone: An IRAK1 inhibitor with in vivo and in vitro anti-inflammatory activities. Sci Rep, 2017. 7: p. 42995.
217. Arjumand, S., et al., Thymoquinone attenuates rheumatoid arthritis by downregulating TLR2, TLR4, TNF-alpha, IL-1, and NFkappaB expression levels. Biomed Pharmacother, 2019. 111: p. 958-963.
218. Haq, A., et al., Nigella sativa: effect on human lymphocytes and polymorphonuclear leukocyte phagocytic activity. Immunopharmacology, 1995. 30(2): p. 147-55.
219. Haq, A., et al., Immunomodulatory effect of Nigella sativa proteins fractionated by ion exchange chromatography. Int J Immunopharmacol, 1999. 21(4): p. 283-95.
220. Al-Ankari, A. S., Immunomodulating effects of black seed and oxytetracycline in pigeons. Immunopharmacol Immunotoxicol, 2005. 27(3): p. 515-20.
221. Salem, M. L., Immunomodulatory and therapeutic properties of the Nigella sativa L. seed. Int Immunopharmacol, 2005. 5(13-14): p. 1749-70.
222. Majdalawieh, A. F., R. Hmaidan, and R. I. Carr, Nigella sativa modulates splenocyte proliferation, Th1/Th2 cytokine profile, macrophage function and NK anti-tumor activity. J Ethnopharmacol, 2010. 131(2): p. 268-75.
223. Boskabady, M. H., et al., Potential immunomodulation effect of the extract of Nigella sativa on ovalbumin sensitized guinea pigs. J Zhejiang Univ Sci B, 2011. 12(3): p. 201-9.
224. Abel-Salam, B. K., Immunomodulatory effects of black seeds and garlic on alloxan-induced Diabetes in albino rat. Allergol Immunopathol (Madr), 2012. 40(6): p. 336-40.
225. Elmowalid, G., A. M. Amar, and A. A. Ahmad, Nigella sativa seed extract: 1. Enhancement of sheep macrophage immune functions in vitro. Res Vet Sci, 2013. 95(2): p. 437-43.
226. Gholamnezhad, Z., M. H. Boskabady, and M. Hosseini, Effect of Nigella sativa on immune response in treadmill exercised rat. BMC Complement Altern Med, 2014. 14: p. 437.
227. Majdalawieh, A. F. and M. W. Fayyad, Immunomodulatory and anti-inflammatory action of Nigella sativa and thymoquinone: A comprehensive review. Int Immunopharmacol, 2015. 28(1): p. 295-304.
228. Kheirouri, S., V. Hadi, and M. Alizadeh, Immunomodulatory Effect of Nigella sativa Oil on T Lymphocytes in Patients with Rheumatoid Arthritis. Immunol Invest, 2016. 45(4): p. 271-83.
229. Swamy, S. M. and B. K. Tan, Cytotoxic and immunopotentiating effects of ethanolic extract of Nigella sativa L. seeds. J Ethnopharmacol, 2000. 70(1): p. 1-7.
230. Salem, M. L., F. Q. Alenzi, and W. Y. Attia, Thymoquinone, the active ingredient of Nigella sativa seeds, enhances survival and activity of antigen-specific CD8-positive T cells in vitro. Br J Biomed Sci, 2011. 68(3): p. 131-7.
231. Ait Mbarek, L., et al., Anti-tumor properties of blackseed (Nigella sativa L.) extracts. Braz J Med Biol Res, 2007. 40(6): p. 839-47.
232. Amara, A. A., M. H. El-Masry, and H. H. Bogdady, Plant crude extracts could be the solution: extracts showing in vivo antitumorigenic activity. Pak J Pharm Sci, 2008. 21(2): p. 159-71.
233. Banerjee, S., et al., Review on molecular and therapeutic potential of thymoquinone in cancer. Nutr Cancer, 2010. 62(7): p. 938-46.
234. Khan, M. A., et al., Anticancer activities of Nigella sativa (black cumin). Afr J Tradit Complement Altern Med, 2011. 8(5 Suppl): p. 226-32.
235. Woo, C. C., et al., Thymoquinone: potential cure for inflammatory disorders and cancer. Biochem Pharmacol, 2012. 83(4): p. 443-51.
236. Lei, X., et al., Thymoquinone inhibits growth and augments 5-fluorouracil-induced apoptosis in gastric cancer cells both in vitro and in vivo. Biochem Biophys Res Commun, 2012. 417(2): p. 864-8.
237. Majdalawieh, A. F. and M. W. Fayyad, Recent advances on the anti-cancer properties of Nigella sativa, a widely used food additive. J Ayurveda Integr Med, 2016. 7(3): p. 173-180.
238. Majdalawieh, A. F., M. W. Fayyad, and G. K. Nasrallah, Anti-cancer properties and mechanisms of action of thymoquinone, the major active ingredient of Nigella sativa. Crit Rev Food Sci Nutr, 2017. 57(18): p. 3911-3928.
239. Mostofa, A. G. M., et al., Thymoquinone as a Potential Adjuvant Therapy for Cancer Treatment: Evidence from Preclinical Studies. Front Pharmacol, 2017. 8: p. 295.
240. Asaduzzaman Khan, M., et al., Thymoquinone, as an anticancer molecule: from basic research to clinical investigation. Oncotarget, 2017. 8(31): p. 51907-51919.
241. Imran, M., et al., Thymoquinone: A novel strategy to combat cancer: A review. Biomed Pharmacother, 2018. 106: p. 390-402.
242. Zhang, Y., et al., Thymoquinone inhibits the metastasis of renal cell cancer cells by inducing autophagy via AMPK/mTOR signaling pathway. Cancer Sci, 2018. 109(12): p. 3865-3873.
243. Ulasli, M., et al., The effects of Nigella sativa (Ns), Anthemis hyalina (Ah) and Citrus sinensis (Cs) extracts on the replication of coronavirus and the expression of TRP genes family. Mol Biol Rep, 2014. 41(3): p. 1703-11.
244. Ahmad, A., et al., A review on therapeutic potential of Nigella sativa: A miracle herb. Asian Pac J Trop Biomed, 2013. 3(5): p. 337-52.
245. Alemi, M., et al., Anti-inflammatory effect of seeds and callus of Nigella sativa L. extracts on mix glial cells with regard to their thymoquinone content. AAPS PharmSciTech, 2013. 14(1): p. 160-7.
246. Shuid, A. N., et al., Nigella sativa: A Potential Antiosteoporotic Agent. Evid Based Complement Alternat Med, 2012. 2012: p. 696230.
247. El Mezayen, R., et al., Effect of thymoquinone on cyclooxygenase expression and prostaglandin production in a mouse model of allergic airway inflammation. Immunol Lett, 2006. 106(1): p. 72-81.
248. Chehl, N., et al., Anti-inflammatory effects of the Nigella sativa seed extract, thymoquinone, in pancreatic cancer cells. HPB (Oxford), 2009. 11(5): p. 373-81.
249. Alkharfy, K. M., et al., The protective effect of thymoquinone against sepsis syndrome morbidity and mortality in mice. Int Immunopharmacol, 2011. 11(2): p. 250-4.
250. Shen, G., et al., Chemoprevention of familial adenomatous polyposis by natural dietary compounds sulforaphane and dibenzoylmethane alone and in combination in ApcMin/+ mouse. Cancer Res, 2007. 67(20): p. 9937-44.
251. Zambrano, V., R. Bustos, and A. Mahn, Insights about stabilization of sulforaphane through microencapsulation. Heliyon, 2019. 5(11): p. e02951.
252. Steinkellner, H., et al., Effects of cruciferous vegetables and their constituents on drug metabolizing enzymes involved in the bioactivation of DNA-reactive dietary carcinogens. Mutat Res, 2001. 480-481: p. 285-97.
253. Fahey, J. W., Y. Zhang, and P. Talalay, Broccoli sprouts: an exceptionally rich source of inducers of enzymes that protect against chemical carcinogens. Proc Natl Acad Sci USA, 1997. 94(19): p. 10367-72.
254. Solowiej, E., et al., Chemoprevention of cancerogenesis—the role of sulforaphane. Acta Pol Pharm, 2003. 60(1): p. 97-100.
255. Gills, J. J., et al., Sulforaphane prevents mouse skin tumorigenesis during the stage of promotion. Cancer Lett, 2006. 236(1): p. 72-9.
256. Myzak, M. C., et al., Sulforaphane inhibits histone deacetylase in vivo and suppresses tumorigenesis in Apc-minus mice. FASEB J, 2006. 20(3): p. 506-8.
257. Singh, A. V., et al., Sulforaphane induces caspase-mediated apoptosis in cultured PC-3 human prostate cancer cells and retards growth of PC-3 xenografts in vivo. Carcinogenesis, 2004. 25(1): p. 83-90.
258. Wang, L., et al., Targeting cell cycle machinery as a molecular mechanism of sulforaphane in prostate cancer prevention. Int J Oncol, 2004. 24(1): p. 187-92.
259. Pham, N. A., et al., The dietary isothiocyanate sulforaphane targets pathways of apoptosis, cell cycle arrest, and oxidative stress in human pancreatic cancer cells and inhibits tumor growth in severe combined immunodeficient mice. Mol Cancer Ther, 2004. 3(10): p. 1239-48.
260. Thejass, P. and G. Kuttan, Antimetastatic activity of Sulforaphane. Life Sci, 2006. 78(26): p. 3043-50.
261. Fimognari, C. and P. Hrelia, Sulforaphane as a promising molecule for fighting cancer. Mutat Res, 2007. 635(2-3): p. 90-104.
262. Li, Y., et al., Sulforaphane, a dietary component of broccoli/broccoli sprouts, inhibits breast cancer stem cells. Clin Cancer Res, 2010. 16(9): p. 2580-90.
263. Lin, W., et al., Sulforaphane suppressed LPS-induced inflammation in mouse peritoneal macrophages through Nrf2 dependent pathway. Biochem Pharmacol, 2008. 76(8): p. 967-73.
264. Ruhee, R. T., S. Ma, and K. Suzuki, Sulforaphane Protects Cells against Lipopolysaccharide-Stimulated Inflammation in Murine Macrophages. Antioxidants (Basel), 2019. 8(12).
265. Xu, X., et al., Effective treatment of severe COVID-19 patients with tocilizumab. Proc Natl Acad Sci USA, 2020.
266. Liu, F., et al., Prognostic value of interleukin-6, C-reactive protein, and procalcitonin in patients with COVID-19. J Clin Virol, 2020. 127: p. 104370.
267. Aziz, M., R. Fatima, and R. Assaly, Elevated Interleukin-6 and Severe COVID-19: A Meta-Analysis. J Med Virol, 2020.
268. Chen, X., et al., Detectable serum SARS-CoV-2 viral load (RNAaemia) is closely correlated with drastically elevated interleukin 6 (IL-6) level in critically ill COVID-19 patients. Clin Infect Dis, 2020.
269. Zhang, C., et al., The cytokine release syndrome (CRS) of severe COVID-19 and Interleukin-6 receptor (IL-6R) antagonist Tocilizumab may be the key to reduce the mortality. Int J Antimicrob Agents, 2020: p. 105954.
270. Zhang, X., et al., First case of COVID-19 in a patient with multiple myeloma successfully treated with tocilizumab. Blood Adv, 2020. 4(7): p. 1307-1310.
271. McGonagle, D., et al., The Role of Cytokines including Interleukin-6 in COVID-19 induced Pneumonia and Macrophage Activation Syndrome-Like Disease. Autoimmun Rev, 2020: p. 102537.
272. Luo, P., et al., Tocilizumab treatment in COVID-19: A single center experience. J Med Virol, 2020.
273. Ulhaq, Z. S. and G. V. Soraya, Interleukin-6 as a potential biomarker of COVID-19 progression. Med Mal Infect, 2020.
274. Fu, B., X. Xu, and H. Wei, Why tocilizumab could be an effective treatment for severe COVID-19? J Transl Med, 2020. 18(1): p. 164.
275. Liu, B., et al., Can we use interleukin-6 (IL-6) blockade for coronavirus disease 2019 (COVID-19)-induced cytokine release syndrome (CRS)? J Autoimmun, 2020: p. 102452.
276. Eren, E., et al., Sulforaphane Inhibits Lipopolysaccharide-Induced Inflammation, Cytotoxicity, Oxidative Stress, and miR-155 Expression and Switches to Mox Phenotype through Activating Extracellular Signal-Regulated Kinase 1/2-Nuclear Factor Erythroid 2-Related Factor 2/Antioxidant Response Element Pathway in Murine Microglial Cells. Front Immunol, 2018. 9: p. 36.
277. Ma, T., et al., Sulforaphane, a Natural Isothiocyanate Compound, Improves Cardiac Function and Remodeling by Inhibiting Oxidative Stress and Inflammation in a Rabbit Model of Chronic Heart Failure. Med Sci Monit, 2018. 24: p. 1473-1483.
278. Liu, H., et al., Biomarker Exploration in Human Peripheral Blood Mononuclear Cells for Monitoring Sulforaphane Treatment Responses in Autism Spectrum Disorder. Sci Rep, 2020. 10(1): p. 5822.
279. Lopez-Chillon, M. T., et al., Effects of long-term consumption of broccoli sprouts on inflammatory markers in overweight subjects. Clin Nutr, 2019. 38(2): p. 745-752.
280. Qi, T., et al., Sulforaphane exerts anti-inflammatory effects against lipopolysaccharide-induced acute lung injury in mice through the Nrf2/ARE pathway. Int J Mol Med, 2016. 37(1): p. 182-8.
281. Dashwood, R. H., et al., Cancer chemopreventive mechanisms of tea against heterocyclic amine mutagens from cooked meat. Proc Soc Exp Biol Med, 1999. 220(4): p. 239-43.
282. Brown, M. D., Green tea (Camellia sinensis) extract and its possible role in the prevention of cancer. Altern Med Rev, 1999. 4(5): p. 360-70.
283. Banerjee, S., et al., Black tea polyphenols restrict benzopyrene-induced mouse lung cancer progression through inhibition of Cox-2 and induction of caspase-3 expression. Asian Pac J Cancer Prev, 2006. 7(4): p. 661-6.
284. Shimizu, M., Y. Shirakami, and H. Moriwaki, Targeting receptor tyrosine kinases for chemoprevention by green tea catechin, EGCG. Int J Mol Sci, 2008. 9(6): p. 1034-49.
285. Johnson, J. J., H.H. Bailey, and H. Mukhtar, Green tea polyphenols for prostate cancer chemoprevention: a translational perspective. Phytomedicine, 2010. 17(1): p. 3-13.
286. Kim, J. W., A. R. Amin, and D. M. Shin, Chemoprevention of head and neck cancer with green tea polyphenols. Cancer Prev Res (Phila), 2010. 3(8): p. 900-9.
287. Henning, S. M., P. Wang, and D. Heber, Chemopreventive effects of tea in prostate cancer: green tea versus black tea. Mol Nutr Food Res, 2011. 55(6): p. 905-20.
288. Du, G. J., et al., Epigallocatechin Gallate (EGCG) is the most effective cancer chemopreventive polyphenol in green tea. Nutrients, 2012. 4(11): p. 1679-91.
289. Henning, S. M., et al., Phenolic acid concentrations in plasma and urine from men consuming green or black tea and potential chemopreventive properties for colon cancer. Mol Nutr Food Res, 2013. 57(3): p. 483-93.
290. Schramm, L., Going Green: The Role of the Green Tea Component EGCG in Chemoprevention. J Carcinog Mutagen, 2013. 4(142): p. 1000142.
291. Rahmani, A. H., et al., Implications of Green Tea and Its Constituents in the Prevention of Cancer via the Modulation of Cell Signalling Pathway. Biomed Res Int, 2015. 2015: p. 925640.
292. Lin, Y. L. and J. K. Lin, (−)-Epigallocatechin-3-gallate blocks the induction of nitric oxide synthase by down-regulating lipopolysaccharide-induced activity of transcription factor nuclear factor-kappaB. Mol Pharmacol, 1997. 52(3): p. 465-72.
293. Jiang, J., et al., Epigallocatechin-3-gallate prevents TNF-alpha-induced NF-kappaB activation thereby upregulating ABCA1 via the Nrf2/Keap1 pathway in macrophage foam cells. Int J Mol Med, 2012. 29(5): p. 946-56.
294. Aneja, R., et al., Epigallocatechin, a green tea polyphenol, attenuates myocardial ischemia reperfusion injury in rats. Mol Med, 2004. 10(1-6): p. 55-62.
295. Xu, Z., et al., Epigallocatechin-3-gallate-induced inhibition of interleukin-6 release and adjustment of the regulatory T/T helper 17 cell balance in the treatment of colitis in mice. Exp Ther Med, 2015. 10(6): p. 2231-2238.

1. A method of treating major depressive disorder comprising stimulation of adult neurogenesis in a mammal.

2. The method of claim 1, wherein said adult neurogenesis is stimulated by administration of human chorionic gonadotropin.

3. The method of claim 1, wherein said adult neurogenesis is stimulated by administration of a nutraceutical composition.

4. The method of claim 3, wherein said nutraceutical composition contains a mixture containing one or more of: a) pterostilbene; b) sulforaphane; c) green tea extract and; d) Nigella sativa.

5. The method of claim 3, wherein said nutraceutical composition is QuadraMune™.

6. The method of claim 2, wherein said nutraceutical is administered together with an anti-inflammatory agent.

7. The method of claim 6, wherein said anti-inflammatory agent is minocycline.

8. The method of claim 2, wherein the drug chlorpromazine is added said nutraceutical.

9. The method of claim 2, wherein the drug haloperidol is added said nutraceutical.

10. The method of claim 2, wherein the drug perphenazine is added said nutraceutical.

11. The method of claim 2, wherein the drug perphenazine is added said nutraceutical.

12. The method of claim 2, wherein the drug fluphenazine is added said nutraceutical.

13. The method of claim 2, wherein the drug clozapine is added said nutraceutical.

14. The method of claim 2, wherein the drug risperidone is added said nutraceutical.

15. The method of claim 2, wherein the drug olanzapine is added said nutraceutical.

16. The method of claim 2, wherein the drug quetiapine is added said nutraceutical.

17. The method of claim 2, wherein the drug ziprasidone is added said nutraceutical.

18. The method of claim 2, wherein the drug aripiprazole is added said nutraceutical.

19. The method of claim 2, wherein the drug paliperidone is added said nutraceutical.

20. The method of claim 2, wherein said nutraceutical is administered together with an inhibitor of NF-kappa B.

21. The method of claim 20, wherein said NF-kappa B inhibitor is selected from a group comprising of: NF-kappa B activity is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2C1), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).
